(19) United States
(12) Patent Application Publication
(10) Pub. No.: US 2007/0135720 A1
(43) Pub. Date: Jun. 14, 2007

(54) BLOOD PRESSURE CUFF WITH CYLINDRICAL BLADDER

(75) Inventor: Simeon M. Vinocur, Portland, OR (US)

Correspondence Address: Simeon M. Vinocur, 3131 SE Tolman Street, Portland, OR, 97202-8662, US

(73) Assignee: LATEX MEDICAL DEVICES, INC., Harlingen, TX (US)

(21) Appl. No.: 11/164,912

(22) Filed: Dec. 9, 2005

(51) Int. Cl. A61B 5/02

(52) U.S. Cl. 600/499; 600/490

(57) ABSTRACT

A generally cylindrical blood pressure bladder encased by a blood pressure cuff. The cylindrical bladder may be either latex or latex-free with one or more tubes to attach to an inflation/deflation device and a measurement instrument. The cylindrical bladder may be sized to fit over the arm, thigh, or finger. The cylindrical bladder may be slightly trapezoidal in shape to serve as a one-size-fits-all. The blood pressure cuff covers the bladder to secure it over the limb, apply proper pressure on the limb, and allow quick deflation of the cylindrical bladder. The blood pressure cuff may be custom fitted for home-use (especially self-applied), office/hospital use, or as part of an automatic machine.

### BACKGROUND OF THE INVENTION

There are many factors that may lead to inaccurate blood pressure measurements. This invention relates to a blood pressure bladder design that improves the accuracy and repeatability of the blood pressure measurement.

The conventional blood pressure apparatus is comprised of a generally rectangular latex blood pressure bladder with either one or two tubes to attach to an inflation/deflation device and a measurement instrument. The conventional blood pressure bladder is encased in a soft nylon cuff and is secured around the limb with Velcro®. Blood pressure measurements can be taken with the blood pressure cuff around the upper arm over the brachial artery, around the thigh over the popliteal artery, or around a finger over the digital artery. The size of the conventional blood pressure bladder is critical to measurement accuracy with a typical width of 40% of arm circumference and a length of 80% of arm circumference.

The conventional method of measuring blood pressure is by placing and securing the blood pressure bladder over the artery, inflating the blood pressure bladder above systolic pressure, then slowly deflating it about 2 mmHg per second. When systolic pressure in the artery exceeds the cuff pressure, blood flows through with each heartbeat and tapping or Korotkoff sounds are heard. These Korotkoff sounds may be heard with a stethoscope over the artery (manual auscultatory method), with a microphone or transducer over the artery (automated or ambulatory method), or as pulses in airflow as the artery flexes (oscillometric method). Blood pressure is measured manually with a mercury manometer or an aneroid manometer. Automated or semi-automated equipment require electronic circuitry.

A common problem with the conventional blood pressure apparatus is that because the rectangular bladder does not cover the entire circumference of the arm, air pressure around the arm is not uniformly distributed, making orientation of the bladder over the artery critical.

To resolve this problem, cylindrical bladders have been developed.

### BRIEF SUMMARY OF THE INVENTION

This invention is a generally cylindrical blood pressure bladder encased by a blood pressure cuff as a replacement for the conventional rectangular blood pressure bladder. The cylindrical bladder may have one or more tubes and be slightly trapezoidal in shape to serve as a one-size-fits-all.

There are three basic embodiments whereby the cylindrical bladder is in a cuff designed for use in a manual auscultatory method, automated or ambulatory method, and oscillometric method.

There are three basic markets for these embodiments, namely: home-use (especially self-applied), office/hospital use, or as part of an automatic machine.

### BRIEF DESCRIPTION OF THE DRAWINGS

FIG. 1 shows how the cylindrical blood pressure bladder slides on the arm.

FIG. 2 shows a side view of the cylindrical bladder.

FIG. 3 shows a bottom view of the cylindrical bladder.

FIG. 4 shows another view posterior of the cylindrical bladder.

FIG. 5 shows a bottom view of the inflated cylindrical bladder.

FIG. 6 shows a functional prototype as it is being applied.

FIG. 7 shows a bottom view of cylindrical bladder and cuff assembly functional prototype.

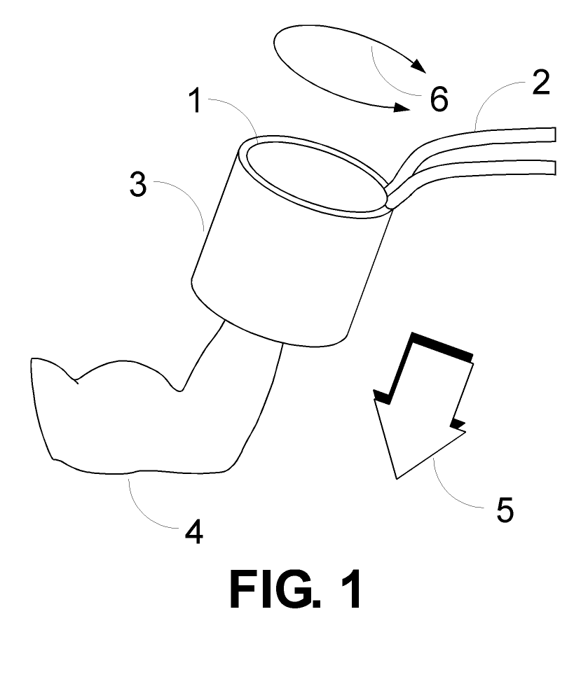

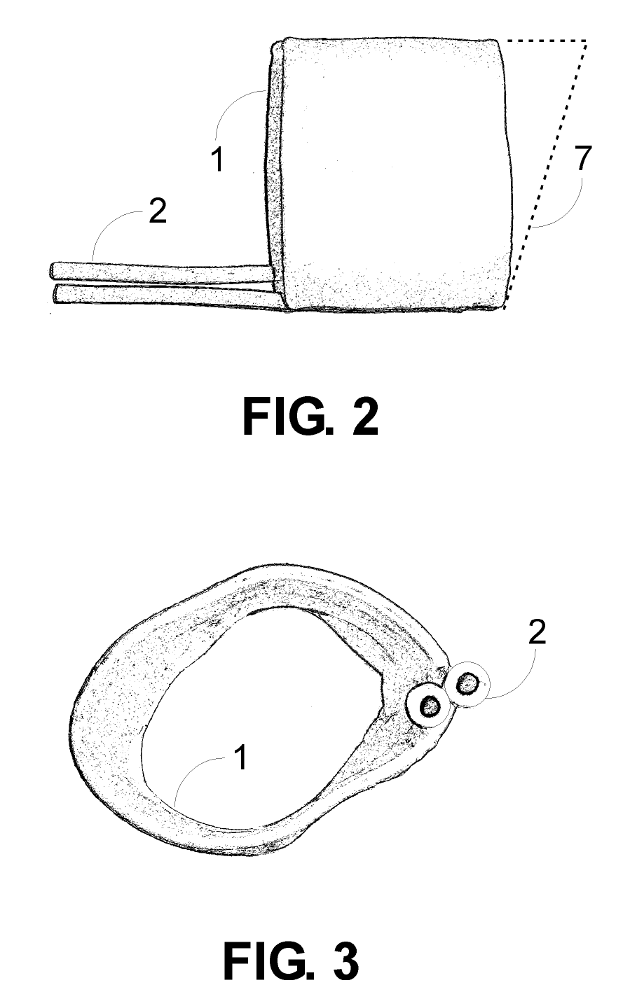

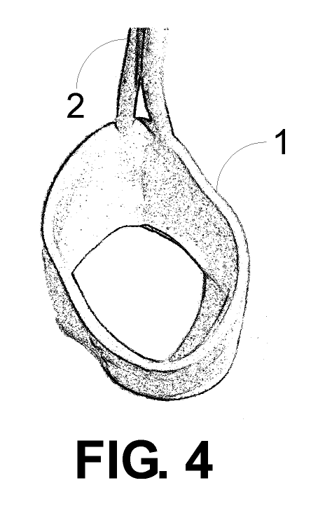

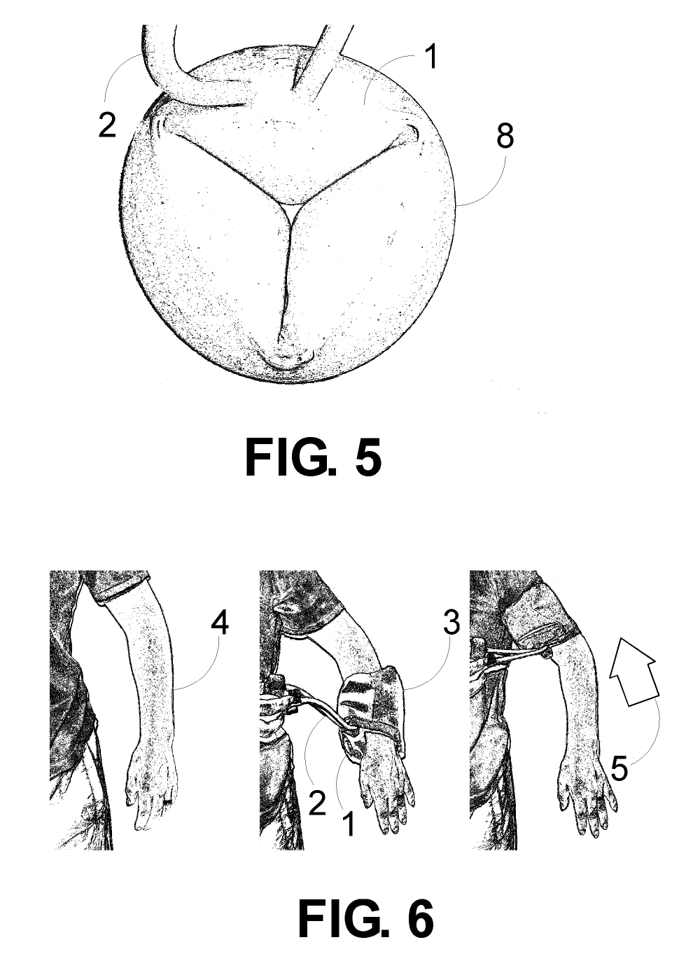

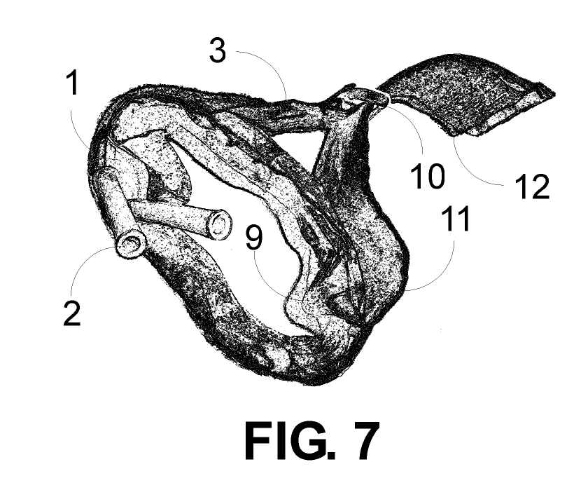

### DETAILED DESCRIPTION OF THE INVENTION

The generally cylindrical blood pressure bladder 1 may have one or more tubes (the Figures shows two tubes 2) to attach to an inflation/deflation device and a measurement instrument. The cylindrical bladder can be made out of latex using a dipping process or may even be latex-free.

The cylindrical bladder is encased in a blood pressure cuff 3. The blood pressure cuff covers the bladder to secure it over the limb, apply proper pressure on the limb, and allow quick deflation of the cylindrical bladder. The blood pressure cuff may be custom fitted for home-use (self-applied), office/hospital use, or as part of an automatic machine.

The cylindrical bladder may be sized to fit the arm 4, leg, or finger and is applied by sliding over 5 the limb. The cylindrical bladder provides more uniform pressure around the limb so that rotational orientation 6 of the bladder over the artery to avoid dead spots is not required.

The side view of the cylindrical bladder may be trapezoidal in shape (more specifically one side wider that the other) 7 to serve as a one-size-fits-all. With this construction, rotation would matter, and the tube(s) would have different orientations to fit small, standard, or obese. When the cylindrical bladder is inflated without the cuff, it becomes generally toroidal in shape 8.

The blood pressure cuff well suited for home use is made of a thin fabric 9 sewn in a cylindrical shape completely covering the blood pressure bladder. The fabric is holds the bladder and keeps the bladder from directly touching the arm, leg or finger. Partially sewn to this fabric is a rectangular soft vinyl covering. On one end of the vinyl covering, an oblong cinch ring 10 is sewn in place. The middle section of the outer vinyl covering has a rectangular pad of the soft Velcro® loop 11. On the same outer side but the other end of the vinyl covering there is a rectangular pad of hard Velcro® hook 12. This end is passed though the cinch ring and attaches to the Velcro® loop (soft) to secure the bladder and cuff around the limb.

This invention is a replacement for the conventional rectangular blood pressure bladder and with the appropriate cuff can be a drop-in replacement for any blood pressure apparatus.

What is claimed is:

1. A blood pressure bladder comprised of an inflatable latex or latex-free air pocket, which is generally cylindrical in shape, becoming generally toroidal in shape when inflated, with one or more tubes attached to it, and constrained externally by a blood pressure cuff.

2. A blood pressure bladder comprised of an inflatable latex or latex-free air pocket, which is of any three dimensional closed geometric shape (including but not limited to a generally cylindrical shape) that encircles the limb, with one or more tubes attached to it, and constrained externally by a blood pressure cuff.

3. The blood pressure bladder of claim 2 wherein the cuff is designed for use in a manual auscultatory method.

4. The blood pressure bladder of claim 2 wherein the cuff is designed for use in an automated or ambulatory method.

5. The blood pressure bladder of claim 2 wherein the cuff is designed for use in an oscillometric method.

6. The blood pressure bladder of claim 2 with cuff designed specifically for home-use.

7. The blood pressure bladder of claim 2 with cuff designed specifically for the physician's office.

8. The blood pressure bladder of claim 2 with cuff designed specifically for hospital-use.

9. The blood pressure bladder of claim 2 sized to slide over the upper arm.

10. The blood pressure bladder of claim 2 sized to slide over the thigh.

11. The blood pressure bladder of claim 2 sized to slide over a finger.

12. A blood pressure bladder of claim 2, whose closed geometric shape provides more uniform pressure around the limb so that rotational orientation of the bladder over the artery to avoid dead spots is not required.

13. The blood pressure cuff of claim 2 constructed of a fabric sheath completely encasing the cylindrical blood pressure bladder and partially attached to a rectangular soft vinyl outer with a standard cinch ring on one end and Velcro® loop and hook attached to the outer part of the vinyl.

14. The blood pressure bladder of claim 2 whose side view is trapezoidal in shape (more specifically one side wider that the other) to serve as a one-size-fits-all.

15. The blood pressure bladder and cuff of claim 2 designed as a drop-in replacement for any blood pressure apparatus.
